(19) United States
(12) Patent Application Publication
(10) Pub. No.: US 2020/0060900 A1
(43) Pub. Date: Feb. 27, 2020

(54) Under Garment with Attached Sanitary Napkin

(71) Applicants: Tyree Sellers, III, Old Hickory, TN (US); Stephanie Sellers, Old Hickory, TN (US)

(72) Inventors: Tyree Sellers, III, Old Hickory, TN (US); Stephanie Sellers, Old Hickory, TN (US)

(21) Appl. No.: 16/529,127

(22) Filed: Aug. 1, 2019

Related U.S. Application Data
(60) Provisional application No. 62/721,845, filed on Aug. 23, 2018.

(51) Int. Cl. A61F 13/84; A61F 13/496; A61F 13/551

(52) U.S. Cl. CPC A61F 13/84; A61F 2013/8476; A61F 13/55145; A61F 13/496

(57) ABSTRACT

An under garment and a container that may be removably attached to the under garment, configured to hold sanitary napkins. The container may be attached via a perforated connection on at least one side thereof. The container may have a first wall and a second wall defining an interior volume therein. In the under garment may be a diaper. In an embodiment there may be sanitary napkins placed within the interior volume of the container.

### CROSS REFERENCE TO RELATED APPLICATIONS

This application claims the benefit of U.S. Provisional Application No. 62/721,845 filed on Aug. 23, 2018. The above identified patent application is herein incorporated by reference in its entirety to provide continuity of disclosure.

### BACKGROUND OF THE INVENTION

The present invention relates to an under garment with attached sanitary napkins. More particularly, the present invention provides for an under garment where there is a package attached to the garment to allow for cleaning of an individual and removal of the package.

Many individuals of all ages wear disposable under garments or diapers. Many times with a diaper comes the need to clean one's self after use or for a parent to do the same. This means that many times extra diapers and cleaning implements must be carried around and available for use. In many instances this means having to remember many different items. Not only does this require multiple different items but these items can take up a considerable amount of space causing the need for a large bag of sorts to carry the items. Many people may accidentally forget extra diapers or wet wipes and be unable to change a soiled diaper.

Consequently, there is a need in for an improvement in the art of disposable under garments and sanitary napkins. The present invention substantially diverges in design elements from the known art while at the same time solves a problem many people face when having to carry around and use such garments and cleaning implements. In this regard the present invention substantially fulfills these needs.

### SUMMARY OF THE INVENTION

In view of the foregoing disadvantages inherent in the known types of under garments and sanitary napkins now present in the prior art, the present invention provides a under garment with attached sanitary napkins wherein the same can be utilized for providing convenience for the user when having to carry around extra under garments and sanitary napkins for when the time arises. The present system comprises A container for holding sanitary napkins including an under garment and a container that may be removably attached to the under garment. The container may be attached via a perforated connection on at least one side of the container. The container may have a first wall and a second wall defining an interior volume therein.

Other objects, features and advantages of the present invention will become apparent from the following detailed description taken in conjunction with the accompanying drawings.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a front/rear view of a under garment with a package of sanitary napkins attached.

FIG. 2a shows a front/rear view of an embodiment of the under garment with a package of sanitary napkins attached.

FIG. 2b shows a front/rear view of an embodiment of the under garment with a package of sanitary napkins attached.

FIG. 2c shows a front/rear view of an embodiment of the under garment with a package of sanitary napkins attached.

FIG. 3a shows a top down view of the sanitary napkin package with a sealing strip at one end.

FIG. 3b shows a top down view of the sanitary napkin package with a sealable opening.

FIG. 4a shows a top down view of an embodiment of the sanitary napkin package where the napkins are stacked therein.

FIG. 4b shows a top down view of an embodiment of the sanitary napkin package where the napkins are rolled therein.

FIG. 4c shows a top down view of an embodiment of the sanitary napkin package where the napkins are attached to each other therein.

### DETAILED DESCRIPTION OF THE INVENTION

Reference is made herein to the attached drawings. Like reference numerals are used throughout the drawings to depict like or similar elements of the under garment with attached sanitary napkins. For the purposes of presenting a brief and clear description of the present invention, a preferred embodiment will be discussed as used for under garment with attached sanitary napkins. The figures are intended for representative purposes only and should not be considered to be limiting in any respect.

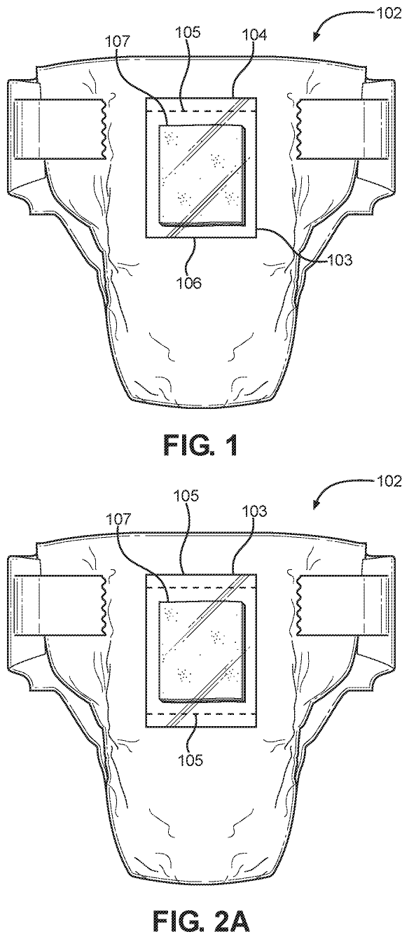

Referring now to FIG. 1, there is shown a front/rear view of a under garment with a package of sanitary napkins attached. There is an under garment 102 with an attached container 103 for holding sanitary napkins 107. The under garment may be a diaper or pull up. One of ordinary skill in the art will understand that a diaper has an adjustable waist for band to adequately fit a person. Further, a diaper has two leg holes with a body therebetween that is substantially water proof in order to contain waste. Lastly, the diaper may be configured in any necessary size or shape to fit various humans of all shapes ages and sizes.

The container has at least a first portion 104 attached by a perforated section 105 to a second portion of the container 106. This perforated section 105 can be used to allow the sanitary napkin container 103 to be torn away from the under garment 102. The first portion 104 of the container can be attached to the under garment 102 in any number of ways. In one embodiment the first portion 104 can be adhesively attached to the under garment 102. This adhesive will allow the container to be attached to the under garment in a way that will reduce the chances that the undergarment is torn when removing the container. In a second embodiment the first portion of the container 104 can be part of the under garment such that a small flap is present to allow for the container to attach to the under garment.

The container 103 may have a first side and a second side that defines a volume therebetween. There may be sanitary napkins 107 placed in the container. These sanitary napkins may be dry napkins or wet. In the event of a wet napkin the napkin may be wet with a cleaning or anti-bacterial solution.

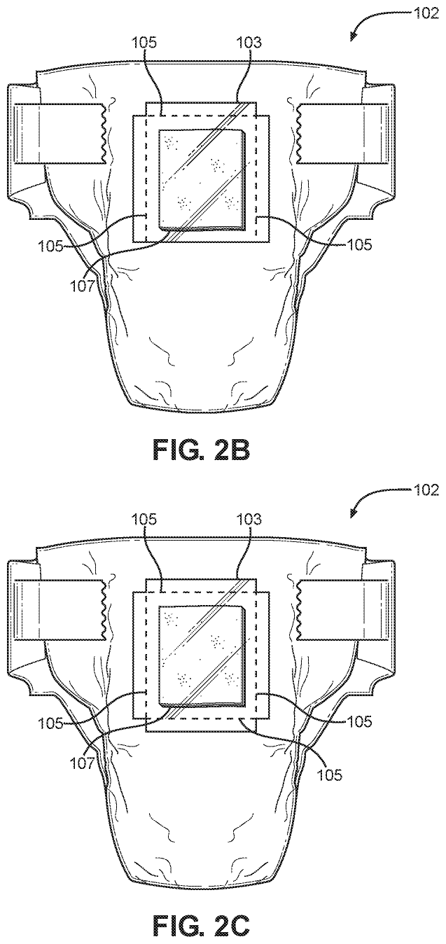

In FIG. 2a-2c there are shown various embodiments where the sanitary napkin container 103 can have various attachments can be on different sides of the container. These attachments can each be seen to have a perforated section 105 on each attachment point. Referring now to FIG. 2a, there is shown a front/rear view of an embodiment of the under garment with a package of sanitary napkins attached. The container is attached to the under garment using two perforated sections. In the shown embodiment the perforated sections are at opposite ends. This will allow the container to be held down at two different sections and avoid becoming disconnected accidentally.

Referring now to FIG. 2b, there is shown a front/rear view of an embodiment of the under garment with a container of sanitary napkins attached. The container 103 is attached to the under garment at three sides all using a perforated attachment. This will allow the container to be securely attached to the under garment. Further, in one embodiment there may be an opening on the fourth side to allow for the wipes to be removed before the container is removed.

Referring now to FIG. 2c, there is shown a front/rear view of an embodiment of the under garment with a package of sanitary napkins attached. The container 103 is attached to four sides of the container. In this embodiment the container will be the most secure to the under garment. In some embodiments the container will have to be completely removed before the wipes can be accessed.

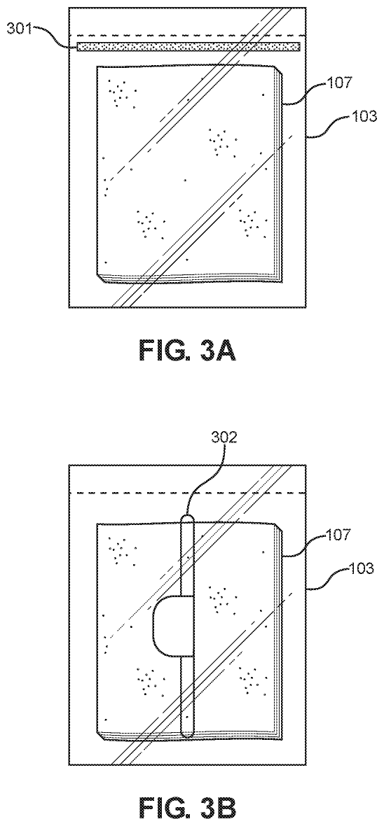

In FIGS. 3a and 3b different embodiments of openings the container may have to allow for sanitary napkins 107 to be removed. Referring now to FIG. 3a, there is shown a top down view of the sanitary napkin package with a sealing strip 301 at one end. In this embodiment the container can be sealed with either an adhesive substance or a channel and lock system. In this embodiment the user can open the container and remove the sanitary napkins therein. In some embodiments the container can be resealed and saved to keep extra napkins.

In the embodiments above the seal can be protected by placing it behind one of the perforated sections 105 between the first and second sides. This will mean that the seal can only be separated after the container is removed from the under garment. This can help to ensure that the container is not opened by mistake and the sanitary napkins allowed to dry out.

Referring now to FIG. 3b, there is shown a top down view of the sanitary napkin container 103 with a sealable opening 302. In this embodiment the opening can be placed one the top of the first or second sides. This opening can be a peel away tab using an adhesive. In this embodiment the adhesive can be such that the tab can be placed back in position and reseal the container. In this embodiment the user will be able to access the sanitary napkins 107 without removing the container from the under garment. This can permit a user to remove a single sanitary napkin if needed and keep the container attached to the under garment.

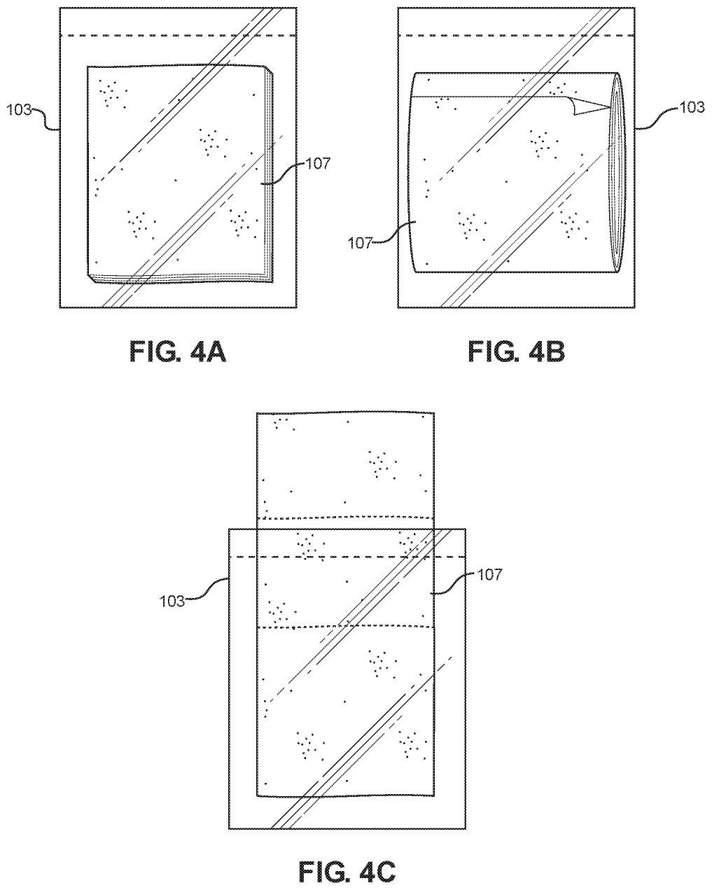

In FIGS. 4a-4c there are shown various embodiments of how a sanitary napkin, or more than one napkin, may be placed within the container 103. Referring now to FIG. 4a, there is shown a top down view of an embodiment of the sanitary napkin container where the sanitary napkins 107 are stacked therein. From the stacked configuration it can be easier to remove a singe napkin and reseal the container. This can also allow for a flatter configuration in which the napkins will not create a bulky package for carrying.

Referring now to FIG. 4b, there is shown a top down view of an embodiment of the sanitary napkin container where the napkins 107 are rolled therein. In this configuration sanitary napkins 107 can be removed from the package one at a time or by the entire roll. In this embodiment the roll may allow for the sanitary napkins to better hold moister from a cleaning solution.

Referring now to FIG. 4c, there is shown a top down view of an embodiment of the sanitary napkin container where the napkins 107 are attached to each other therein. In this embodiment a sanitary napkin 107 can be taken from the container and torn from the others. This will allow for the next napkin to be readily available to be grabbed when needed.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

We claim:

1) A container for holding sanitary napkins comprising:
an under garment;
a container removably attached to the under garment, wherein the container is attached via a perforated connection on at least one side;
the container having at least a first wall and a second wall defining an interior volume therein.

2) The container for holding sanitary napkins of claim 1, wherein the under garment is a diaper.

3) The container for holding sanitary napkins of claim 1, further comprising sanitary napkins placed within the interior volume of the container.

4) The container for holding sanitary napkins of claim 3, further comprising: an opening on the container to allow for the removal of the sanitary napkins.

5) The container for holding sanitary napkins of claim 4, wherein the opening in operably secured together.

6) The container for holding sanitary napkins of claim 1, wherein the perforated connection is on a top portion and a bottom portion of the container.

7) The container for holding sanitary napkins of claim 1, wherein the container is made out of a flexible plastic.
